(19) United States
(12) Patent Application Publication
(10) Pub. No.: US 2020/0188051 A1
(43) Pub. Date: Jun. 18, 2020

(54) vertical self-service blood pressure detector

(71) Applicant: Hui Jiao, Ningbo City (CN)

(72) Inventor: Hui Jiao, Ningbo City (CN)

(21) Appl. No.: 16/802,505

(22) Filed: Feb. 26, 2020

(30) Foreign Application Priority Data
Oct. 29, 2019 (CN) 201911038113X

(51) Int. Cl. A61B 50/10; A61B 5/022; A61B 5/00; A61B 90/98

(52) U.S. Cl. CPC A61B 50/10; A61B 5/022; A61B 5/702; A61B 2050/3013; A61B 5/7475; A61B 90/98; A61B 2050/105; A61B 5/7435

(57) ABSTRACT

The invention discloses a vertical self-service blood pressure detector, which comprises a vertical cabinet. A control board is fixedly arranged on the right end of the upper end surface of the vertical cabinet, a touch screen is arranged on the left end surface of the control board, and a card reader is arranged on the lower side of the touch screen. The user needs to insert a personal social security card, medical card, etc. into the card reader to read personal information before using the device. The present invention allows the user to automatically use the device by placing the blood pressure detector in a vertical cabinet. The user needs to use a social security card or medical card for real-name use. Not only can the user be recorded, but also the test data can be recorded in the social security card or medical card, which is convenient for doctors to obtain data. The stool is stored, and the blood pressure detector is effectively protected, and it is avoided that someone removes the stool, thereby affecting the user experience.

### CROSS-REFERENCES TO RELATED APPLICATIONS

The present application claims priority from Chinese application No. 201911038113X filed on Sep. 19, 2019 which is hereby incorporated by reference in its entirety.

### TECHNICAL FIELD

The invention relates to the technical field of medical diagnostic instruments, in particular to a vertical self-service blood pressure detector.

### BACKGROUND OF THE INVENTION

In medical institutions, there are many patients seeing a doctor every day. Many people need to wait in line. Some people even wait for a simple blood pressure measurement. This not only greatly wastes the patient's time, but also increases the workload of the doctor. Therefore, a self-service blood pressure monitor is needed. However, because there are many people in the hospital, placing the blood pressure detector directly in the public area not only has the possibility of loss, but also the data cannot be recorded after the test. At the same time, it is usually necessary to sit on a stool during the test, and the stool loss rate is more High, greatly reducing the user experience, and the availability is not high. The present invention illustrates a device capable of solving the above problems.

### BRIEF SUMMARY OF THE INVENTION

### Technical Problem

Placing an ordinary blood pressure monitor directly in a public area is not only easy to lose, but also the data cannot be recorded and the use effect is not good.

In order to solve the above problems, a vertical self-service blood pressure monitor is designed in this example. A vertical self-service blood pressure monitor in this example includes a vertical cabinet, and a control board is fixed to the right end of the upper end of the vertical cabinet and the left end of the control board is fixed. A touch screen is provided in the plane, and a card reader is provided on the lower side of the touch screen. The user needs to insert a personal social security card, medical card, etc. into the card reader to read personal information before using the device. A detector storage device is provided on the upper side of the vertical cabinet and on the left side of the control board. The detector storage device includes an instrument storage cavity provided in the upper end surface of the vertical cabinet, and the instrument storage cavity can slide up and down. A lifting plate is provided, and a blood pressure detector is fixed at the upper end of the lifting plate. A flip plate is symmetrically arranged on the upper side of the instrument storage cavity. When the lifting plate drives the blood pressure detector to rise to the upper side of the vertical cabinet At that time, the flip board is synchronously opened, and an arm can be extended into the blood pressure detector for blood pressure detection. The detected data is displayed on the touch screen and stored to social security through the card reader. Or a medical card, a stool storage cavity is provided on the lower side of the instrument storage cavity, a mobile device is provided on the right side of the stool storage cavity, a mobile plate is provided in the mobile device, and a side of the mobile plate is provided There is a stool. A sealing plate is provided on the left side of the stool storage cavity. Pulling devices are symmetrically arranged on the front and back sides of the stool storage cavity and facing the front and back sides of the sealing plate. The sealing plate is opened. At this time, the seat can be moved to the left of the standing cabinet by the moving plate for use by the inspector.

Preferably, an active slide cavity is connected to the lower wall of the instrument storage cavity, and a telescopic screw is rotatably provided between the left and right walls of the active slide cavity. The telescopic screw is divided into left and right sections and the thread directions are opposite. In the active sliding cavity, there are active sliders that are symmetrically and slidably left and right, which are respectively screwed to the left and right sections of the telescopic screw. The lifting plate is provided with a slave opening that faces downward and directly above the active sliding cavity. A guide slide rod is fixedly connected between the left and right walls of the driven slide cavity, and a driven slider slidingly connected to the guide slide rod is symmetrically and slidably arranged in the driven slide cavity. A telescopic hinge is connected to a hinge between the driven slider and the driving slider.

Preferably, a control motor is fixed in the right wall of the active sliding cavity, and the right end of the telescopic screw is dynamically connected to the control motor.

Preferably, the upper side of the instrument storage cavity is provided with a flap cavity with an upward opening, the flip plate is rotatably connected to the left and right walls of the flap cavity at an end away from the center of symmetry, and the lower end surface of the flip plate is fixedly located at A circular arc rod in the instrument storage cavity, a lower end of the circular arc rod is hinged to a push-pull rod, and a lower end of the push-pull rod is hinged to an upper end of the lifting plate.

Wherein, the moving device includes a translation cavity provided in the moving plate, a translation screw is rotatably provided between the left and right walls of the translation cavity, and a left and right slide in the translation cavity is provided with a screw connection to the translation cavity. The translation block of the translation screw is provided with a support rod chute on the upper side of the translation cavity. The upper end of the translation block is fixed with a support rod that can slide left and right in the support rod chute, and the seat is fixed. The upper end of the support rod is provided with an elastic cushion fixed on the upper end surface of the stool, a bevel gear cavity is provided on the right side of the translation cavity, and a driven gear is fixedly extended to the right end of the translation screw cavity. A bevel gear, a driving bevel gear engaging with the driven bevel gear is rotatably provided between the front and rear walls of the bevel gear cavity, a shaft is fixed on the driving bevel gear axis, and the front and rear walls of the seat receiving cavity A gear chute facing the front and back sides of the bevel gear cavity is symmetrical and connected. The front and rear ends of the rotating shaft extend into the gear chute, and a spur gear is fixed. The lower wall of the gear chute is fixed. There is a rack gear engaged with the lower end of the spur gear.

Preferably, a sprocket cavity is provided on the upper side of the gear chute on the rear side, and a sprocket is provided symmetrically and rotatably between the front and rear walls of the sprocket cavity. A chain, the lower half of the chain can be engaged with the upper end surface of the spur gear, a mobile motor is fixedly located on the right end in the rear wall of the sprocket cavity, and the sprocket on the right side is dynamically connected to the front end of the mobile motor.

Preferably, the lower end surface of the moving plate is provided with a roller cavity facing downwards, and a roller shaft is symmetrically and rotatable between the front and rear walls of the roller cavity. The roller shaft is symmetrical and fixed on the front and back of the outer circle. A roller is provided, which facilitates the movement of the moving plate.

Wherein, the pulling device includes horizontal chute symmetrically and connected to the upper ends of the front and rear walls of the stool storage cavity, and the upper ends of the front and rear ends of the sealing plate are symmetrical and fixedly provided with slideable left and right in the horizontal chute. Water-smooth column, the left end of the front and rear walls of the stool storage cavity are symmetrical and connected with vertical slide grooves, and the lower ends of the front and rear end surfaces of the sealing plate are symmetrical and fixed with vertical slides that can slide up and down in the vertical slide groove A pull cavity is connected to one side of the vertical slide groove away from the stool storage cavity, and a pull block is slidably arranged in the pull cavity. The vertical slide column rotates away from the sealing plate at one end. Connected in the pulling block, a spring is fixedly connected between the upper end of the pulling block and the upper wall of the pulling cavity.

Preferably, a reel cavity is provided on the upper side of the pulling cavity, a reel is rotatably provided between the front and rear walls of the reel cavity, and a rope is wound around the reel. The lower end is fixedly connected to the upper end surface of the pulling block, the reel shaft is fixedly provided with a transmission shaft, a synchronization cavity is provided in the inner wall between the reel chambers, and the opposite end of the transmission shaft extends into the synchronization cavity. A transmission bevel gear is fixedly provided, and the left end of the telescopic screw extends into the synchronization cavity and a power bevel gear meshing with the transmission bevel gear is fixedly provided.

Preferably, a voice player is provided in the left end surface of the control board and on the upper side of the touch screen, and the voice player can guide the user how to use the device by means of voice, and at the same time can broadcast the detection and report. It is helpful for the elderly or those with eyes difficulty to obtain the detection information.

The beneficial effect of the present invention is that by placing the blood pressure detector in a vertical cabinet, the present invention can be used automatically by the user. The user needs to use a social security card or a medical card for real-name use, which can not only record the user, but also Record the test data in the social security card or medical card, which is convenient for doctors to obtain the data. At the same time, the device stores the blood pressure detector and the stool to effectively protect the blood pressure detector, and to prevent someone from removing the stool, which affects User experience.

### BRIEF DESCRIPTION OF THE DRAWINGS

For ease of explanation, the present invention is described in detail by the following specific embodiments and the accompanying drawings.

FIG. 1 is a schematic diagram of the overall structure of a vertical self-service blood pressure detector according to the present invention;

FIG. 2 is a schematic structural diagram of the direction “A-A” of FIG. 1; FIG.

FIG. 3 is an enlarged schematic view of the structure of “B” in FIG. 1; FIG.

FIG. 4 is a schematic structural diagram of the “C-C” direction of FIG. 1; FIG.

FIG. 5 is a schematic structural diagram of the “D-D” direction of FIG. 1; FIG.

FIG. 6 is a schematic structural diagram of the “E-E” direction of FIG. 5.

### DETAILED DESCRIPTION OF THE INVENTION

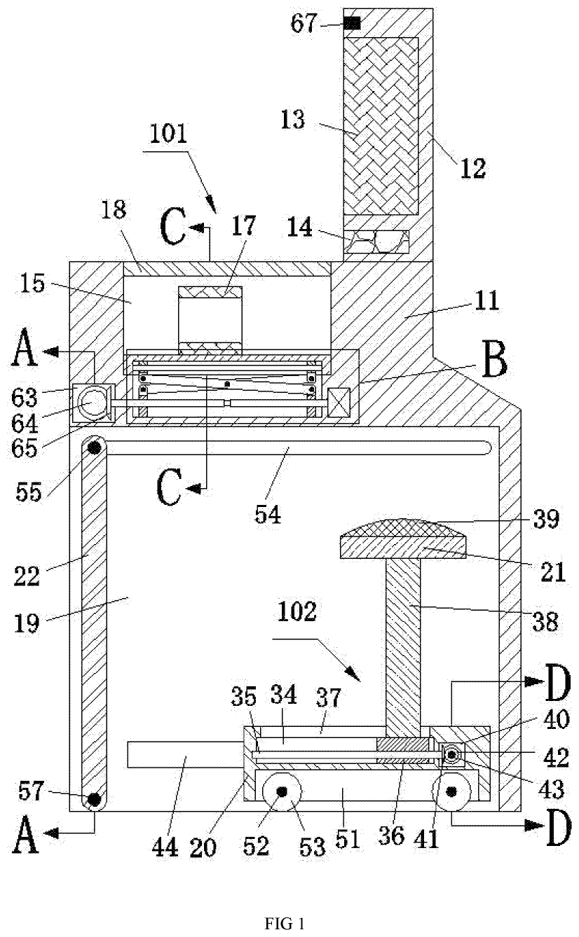

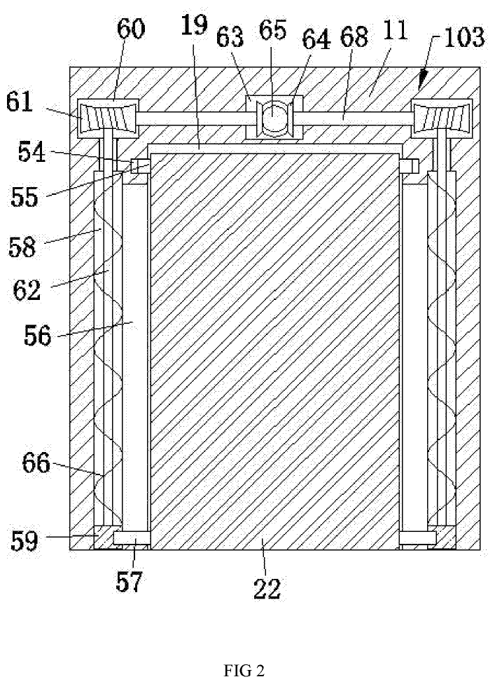

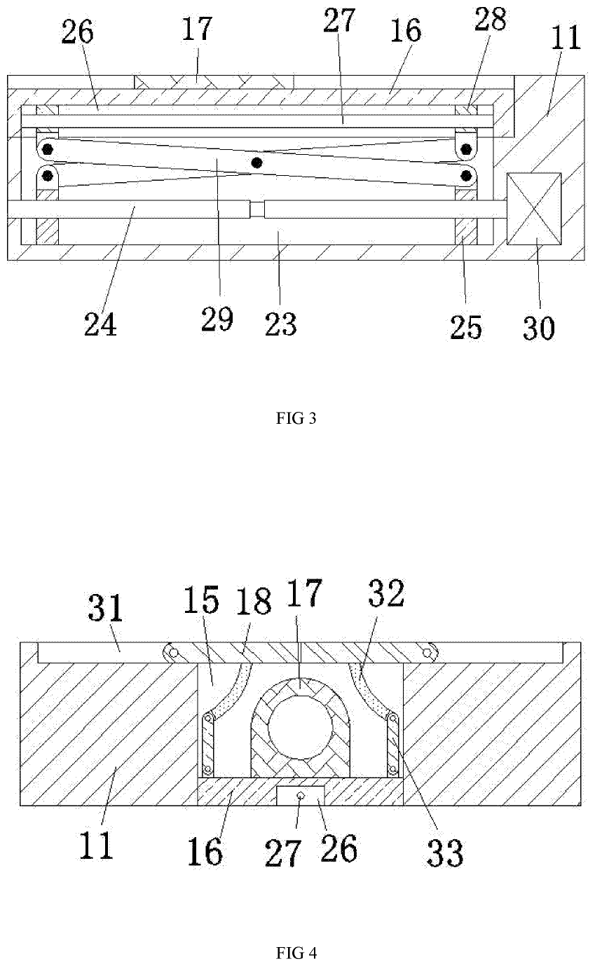

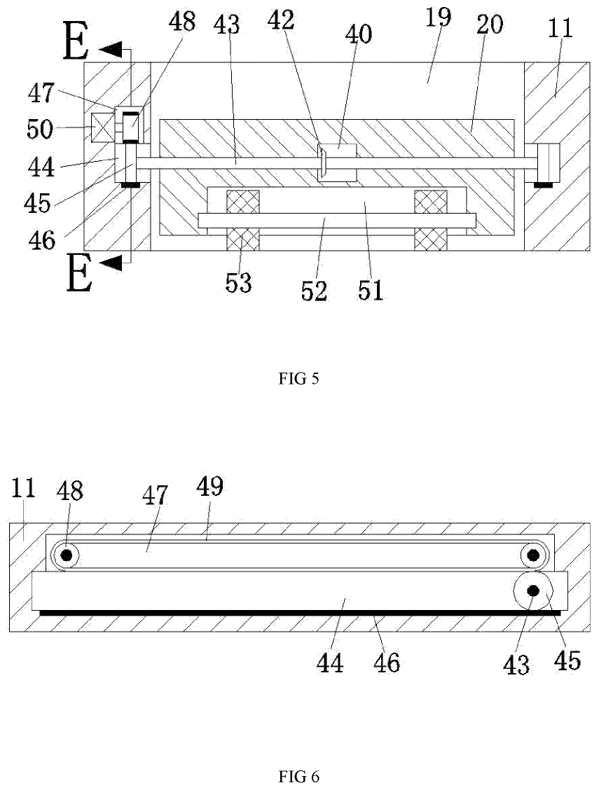

The present invention is described in detail below with reference to FIGS. 1 to 6. For convenience of description, the orientation described below is defined as follows: the up-down, left-right, front-back direction described below is consistent with the up-down, left-right, front-back direction of the projection relationship of FIG.

The invention relates to a vertical self-service blood pressure detector, which is mainly used for blood pressure detection during medical examination. The invention will be further described below with reference to the accompanying drawings of the invention:

The vertical self-service blood pressure detector according to the present invention comprises a vertical cabinet 11, and a control board 12 is fixedly located on the right end of the upper end of the vertical cabinet 11, and a touch screen 13 is arranged on the left end of the control board 12. There is a card reader 14 on the side. The user needs to insert a personal social security card, medical card, etc. into the card reader 14 to read personal information before using the device. The upper side of the vertical cabinet 11 is located in the office. A detector storage device 101 is provided on the left side of the control board 12, and the detector storage device 101 includes an instrument storage cavity 15 provided in the upper end surface of the vertical cabinet 11. There is a lifting plate 16, a blood pressure detector 17 is fixed on the upper end surface of the lifting plate 16, and a flip plate 18 is symmetrically arranged on the upper side of the instrument storage cavity 15. When the lifting plate 16 drives the blood pressure detector 17 to rise When the upper side of the vertical cabinet 11 is opened, the flip plate 18 is synchronously opened. At this time, an arm can be extended into the blood pressure detector 17 for blood pressure detection. The detected data is displayed on the touch screen 13 and read through the reading screen. Card reader 14 stored to social security card or medical Inside the card, a stool storage cavity 19 is provided on the lower side of the instrument storage cavity 15, and a mobile device 102 is provided on the right side of the stool storage cavity 19, and a moving plate 20 is provided in the mobile device 102. A seat 21 is provided on the upper side of the plate 20, and a sealing plate 22 is provided on the left side of the seat storage cavity 19, and the seat storage cavity 19 is symmetrically provided on the front and back sides of the seat storage cavity 19 and facing the front and back sides of the sealing plate 22. The pulling device 103 can drive the sealing plate 22 to open. At this time, the moving plate 20 can be used to move the seat 21 to the left of the standing cabinet 11 for use by the inspector.

Beneficially, the lower wall of the instrument storage cavity 15 is provided with an active slide cavity 23, and a telescopic screw 24 is rotatably provided between the left and right walls of the active slide cavity 23. The telescopic screw 24 is divided into left and right sections and has a thread direction. On the contrary, the active slide cavity 23 is provided with a left-right symmetrical and slidable active slider 25 that is respectively screwed to the left and right sections of the telescopic screw 24. The lifting plate 16 is provided with an opening facing downward and facing directly. The driven sliding chamber 26 above the active sliding chamber 23 is fixedly connected with the guide sliding rod 27 between the left and right walls of the driven sliding chamber 26. The left and right sliding chambers 26 are symmetrically and slidably provided with a sliding block. A driven slider 28 connected to the guide slider 27, and a telescopic hinge 29 is connected to the hinge between the driven slider 28 and the driving slider 25.

Beneficially, a control motor 30 is fixed in the right wall of the active sliding cavity 23, and the right end of the telescopic screw 24 is dynamically connected to the control motor 30.

Beneficially, the upper side of the instrument storage cavity 15 is provided with a flap cavity 31 with an upward opening. The flip plate 18 is rotatably connected to the left and right walls of the flap cavity 31 at an end away from the center of symmetry. A circular arc rod 32 is fixedly located in the instrument storage cavity 15, a lower end of the circular arc rod 32 is hinged with a push-pull rod 33, and a lower end of the push-pull rod 33 is hinged to the upper end surface of the lifting plate 16.

According to an embodiment, the mobile device 102 is described in detail below. The mobile device 102 includes a translation cavity 34 provided in the moving plate 20, and a translation screw 35 is rotatably provided between the left and right walls of the translation cavity 34. A translation block 36 that is threadedly connected to the translation screw 35 is provided in the translation cavity 34 that can be slid left and right, and a support rod chute 37 with an upward opening is provided on the upper side of the translation cavity 34. The upper end of the translation block 36 is fixed. A support rod 38 is provided which can slide left and right in the support rod chute 37. The stool 21 is fixedly disposed on the upper end of the support rod 38. An elastic cushion 39 is fixed on the upper end surface of the stool 21. The right side of the translation cavity 34 is provided with a bevel gear cavity 40, and the right end of the translation screw 35 extends into the bevel gear cavity 40 to be fixed with a driven bevel gear 41. The bevel gear cavity 40 is rotatable between the front and rear walls. There is a driving bevel gear 42 meshing with the driven bevel gear 41. The driving bevel gear 42 is fixedly provided with a rotating shaft 43 at its center. The front and rear walls of the seat receiving cavity 19 are symmetrical and communicate with each other and face the bevel gear. Gear chute 44 on the front and back sides of the cavity 40, and the front and rear ends of the rotation shaft 43 extend to the teeth In the chute 44 is provided with a spur gear 45 fixed to the inner wall of the gear case 44 is fixed chute 45 provided with a lower end engaged with the linear gear rack 46.

Beneficially, a sprocket cavity 47 is provided on the upper side of the gear chute 44 on the rear side, and a sprocket 48 is symmetrically and rotatable between the front and rear walls of the sprocket cavity 47. A chain 49 is interposed, and the lower half of the chain 49 can be engaged with the upper end surface of the spur gear 45. The right end of the rear wall of the sprocket cavity 47 is fixed with a moving motor 50, and the right side of the sprocket 48 is powered by Connected to the front end of the moving motor 50.

Beneficially, the lower end surface of the moving plate 20 is provided with a roller cavity 51 which is opened downward. The roller cavity 51 is provided with a roller shaft 52 symmetrically and rotatably between the front and rear walls. The roller shaft 52 is in front of the outer circle. The rear wheel is symmetrical and fixedly provided with a roller 53 that facilitates the movement of the moving plate 20.

According to the embodiment, the pulling device 103 will be described in detail below. The pulling device 103 includes horizontal slide grooves 54 symmetrically and connected to the upper ends of the front and rear walls of the stool storage cavity 19, and the upper ends of the front and rear ends of the sealing plate 22 are symmetrical. A horizontal sliding column 55 that can be slid left and right in the horizontal sliding groove 54 is fixedly provided. The left end of the front and rear walls of the stool storage cavity 19 is symmetrical and a vertical sliding groove 56 is connected to the lower end of the front and rear end surfaces of the sealing plate 22. A vertical sliding column 57 that can slide up and down in the vertical chute 56 is provided symmetrically and fixedly. A pull cavity 58 is provided on the side of the vertical chute 56 remote from the stool storage cavity 19. A pulling block 59 is provided in the pulling cavity 58 to be able to slide up and down. One end of the vertical sliding column 57 away from the sealing plate 22 is rotatably connected to the pulling block 59. The upper end of the pulling block 59 is connected to the pulling cavity 58. A spring 66 is fixedly connected between the upper walls.

Beneficially, a reel cavity 60 is provided on the upper side of the pulling cavity 58, and a reel 61 is rotatably provided between the front and rear walls of the reel cavity 60, and a rope 62 is wound around the reel 61. The lower end of the pulling rope 62 is fixedly connected to the upper end surface of the pulling block 59, the reel 61 is fixedly provided with a transmission shaft 68 at its axis, and a synchronization cavity 63 is provided in the inner wall between the reel cavity 60. The opposite end of the transmission shaft 68 extends into the synchronization cavity 63 and is fixed with a transmission bevel gear 64, and the left end of the telescopic screw 24 extends into the synchronization cavity 63 and is fixed with a power cone engaged with the transmission bevel gear 64. Gear 65.

Beneficially, a voice player 67 is provided in the left end surface of the control board 12 and on the upper side of the touch screen 13. The voice player 67 can guide the user how to use the device through voice, and at the same time, the detection can be seen. Broadcasting will help the elderly or people with eye discomfort to obtain detection information.

The following describes the steps of using a vertical self-service blood pressure monitor in detail in conjunction with FIG. 1 to FIG. 6:

In the initial state, the lifting plate 16 is in the lower limit position and retracted into the instrument storage cavity 15. The flip plate 18 closes the upper side of the instrument storage cavity 15. The moving plate 20 is in the right limit position and the translation block 36 is in the right limit position. The sealing plate 22 is in a vertical state and closes the left side of the stool storage cavity 19, and the pulling block 59 is in an upper limit position.

When in use, insert the social security card or medical card into the card reader 14, and the user is prompted by the voice player 67 to leave a certain distance from the left side of the cabinet 11 at this time. At this time, the control motor 30 is started and the telescopic screw 24 is rotated, The driving sliders 25 are brought closer to each other through the screw connection, and the driven sliders 28 are brought closer to each other and moved upward through the telescopic hinge 29, and then the lifting plate 16 is moved upward and the blood pressure detector 17 is raised. At this time, the lifting plate 16 is pushed by the pull rod 33 pushes the arc rod 32 upwards, and then drives the opposite end of the flip plate 18 to flip upwards and away from the center of symmetry to a horizontal state. At this time, the lifting plate 16 drives the blood pressure detector 17 to rise to the upper side of the vertical cabinet 11. In the process, the telescopic The screw 24 drives the power bevel gear 65 to rotate, and the transmission bevel gear 64 rotates through the gear meshing. The drive shaft 68 drives the reel 61 to rotate and winds the pull rope 62, and then pulls the pull block 59 to move upward, and then through the vertical The lower end of the sealing plate 22 is moved upward by the sliding column 57. At this time, the horizontal sliding column 55 is slid to the right along the horizontal sliding groove 54, and then the upper end of the sealing plate 22 is moved to the right. At this time, the sealing plate 22 changes to a horizontal state. When the blood pressure detector 17 moves to the upper limit position, the sealing plate 22 is close to the level. At this time, the mobile motor 50 starts and drives the right sprocket 48 to rotate, thereby driving the chain. 49 rotates clockwise, the spur gear 45 on the rear side is rotated counterclockwise by the gear meshing, and the driving bevel gear 42 and the spur gear 45 on the front side are synchronously rotated by the rotating shaft 43. At this time, the spur gear 45 runs along the rack 46 and passes the gear. The meshing moves while rotating to the left, and then drives the moving plate 20 to move to the left. At this time, the driving bevel gear 42 drives the driven bevel gear 41 through the gear meshing and the translation screw 35 to rotate, and the translation block 36 is moved to the left through the screw connection. The seat 21 and the elastic cushion 39 are driven to the left by the support rod 38. When the moving plate 20 is moved to the left extreme position, the stool 21 and the elastic cushion 39 are moved to the left of the stand 11 at this time. You can sit on the elastic cushion 39 and put your hand into the blood pressure monitor 17 for blood pressure detection. The test result is displayed on the touch screen 13 and played by the voice player 67, and counted by the card reader 14. It is recorded in the medical record card or medical card;

After the end of use, when no one continues to insert the card into the card reader 14 within one minute, and the device is left unused by default, the device returns to the initial state.

The beneficial effect of the present invention is that by placing the blood pressure detector in a vertical cabinet, the present invention can be used automatically by the user. The user needs to use a social security card or a medical card for real-name use, which can not only record the user, but also Record the test data in the social security card or medical card, which is convenient for doctors to obtain the data. At the same time, the device stores the blood pressure detector and the stool to effectively protect the blood pressure detector, and to prevent someone from removing the stool, which affects User experience.

In the above manner, those skilled in the art can make various changes according to the working mode within the scope of the present invention.

1. A vertical self-service blood pressure detector includes a vertical cabinet, characterized in that a control board is fixedly arranged on the right end of the upper end surface of the vertical cabinet, a touch screen is arranged on the left end surface of the control board, and a card reader is arranged on the lower side of the touch screen. The user needs to insert a personal social security card, medical card, etc. into the card reader to read personal information before using the device;
a detector storage device is provided on the upper side of the vertical cabinet and on the left side of the control board. The detector storage device includes an instrument storage cavity provided in the upper end surface of the vertical cabinet. A sliding plate is provided, and a blood pressure detector is fixed on the upper surface of the lifting plate. A flip plate is symmetrically arranged on the upper side of the instrument storage cavity. When the lifting plate drives the blood pressure detector to rise to the vertical cabinet When it is on the upper side, the flip board is synchronously opened. At this time, the arm can be extended into the blood pressure detector for blood pressure detection. The detected data is displayed on the touch screen and stored on the social security card or medical treatment through the card reader. Carne
a stool storage cavity is provided on the lower side of the instrument storage cavity, and a moving device is provided on the right side of the stool storage cavity. A moving plate is provided in the moving device, and a stool is provided on the upper side of the moving plate. A sealing plate is provided on the left side of the stool storage cavity;
pulling devices are symmetrically provided on the front and back sides of the stool storage cavity and are opposite to the front and back sides of the sealing plate. The pulling device can drive the sealing plate to open. At this time, the moving plate can be used to drive the sitting stool. Move left to the left side of the stand cabinet for use by the tester.

2. The vertical self-service blood pressure detector according to claim 1, characterized in that the lower wall of the instrument storage cavity is connected with an active sliding cavity, and a telescopic screw is rotatably provided between the left and right walls of the active sliding cavity. The telescopic screw is divided into left and right sections and the directions of the threads are opposite. The active slide cavity is symmetrical and slidable with active sliders that are respectively screwed to the left and right sections of the telescopic screw. A driven sliding cavity is provided with the opening downward and facing directly above the driving sliding cavity. Guide slide rods are fixedly connected between the left and right walls of the driven sliding cavity. The driven sliding cavity is symmetrical and slidable left and right. A driven slider slidingly connected to the guide slider is provided, and a telescopic hinge is connected to the hinge between the driven slider and the active slider.

3. The vertical self-service blood pressure detector according to claim 2, wherein a control motor is fixedly located in the right wall of the active slide cavity, and the right end of the telescopic screw is dynamically connected to the control motor.

4. The vertical self-service blood pressure tester according to claim 1, characterized in that: the upper side of the instrument storage cavity is connected with an upwardly turned flap cavity, and the flip plate is rotatably connected to the end away from the center of symmetry. The left and right walls of the turning plate cavity, the lower end of the turning plate is fixedly provided with a circular arc rod located in the instrument storage cavity, a lower end of the circular arc rod is hinged to a push-pull rod, and a lower end of the push-pull rod is hinged to the lift The upper end of the board.

5. The vertical self-service blood pressure detector according to claim 1, wherein the mobile device comprises a translation cavity provided in the moving plate, and a translation is provided between the left and right walls of the translation cavity. A screw, a translation block that is threadedly connected to the translation screw is slidably left and right in the translation cavity, and a support rod chute with an upward opening is arranged on the upper side of the translation cavity; the left and right sliding rods in the supporting rod chute are fixed on the upper end of the supporting rod, an elastic cushion is fixed on the upper end surface of the supporting stool, and a bevel gear cavity is provided on the right side of the translation cavity. The right end of the translation screw extends into the bevel gear cavity and is provided with a driven bevel gear. A driving bevel gear meshing with the driven bevel gear is rotatably provided between the front and rear walls of the bevel gear cavity. The driving bevel gear axis is fixed with a rotating shaft. The front and back walls of the seat storage cavity are symmetrical and connected with gear chute facing the front and back sides of the bevel gear cavity. The front and back ends of the rotating shaft extend to the gear slide. A spur gear is fixed in the groove, and the gear A rack which is engaged with the lower end of the spur gear is fixed in the lower wall of the chute.

6. The vertical self-service blood pressure detector according to claim 5, characterized in that a sprocket cavity is connected to the upper side of the gear chute on the rear side, and the front and back walls of the sprocket cavity are symmetrical and can be left A sprocket is provided for rotation, and a chain is wound between the sprocket wheels. The lower half of the chain can be engaged with the upper end surface of the spur gear. The sprocket is connected to the front end of the moving motor.

7. The vertical self-service blood pressure detector according to claim 1, characterized in that: a roller chamber with a downward opening is provided in the lower end surface of the moving plate, and the left and right walls of the roller chamber are symmetrical and rotatable. A roller shaft is provided, and the roller shaft is symmetrically and fixedly provided on the front and back of the outer circle, and the roller facilitates the movement of the moving plate.

8. The vertical self-service blood pressure detector according to claim 1, wherein the pulling device comprises horizontal chute symmetrically and connected to the upper ends of the front and rear walls of the stool storage cavity, and the sealing plate The upper end is symmetrical and fixed with horizontal sliding columns that can slide left and right in the horizontal chute. The left ends of the front and rear walls of the stool storage cavity are symmetrical and connected with vertical chute. The lower ends of the front and rear end surfaces of the sealing plate are symmetrical and A vertical sliding column that can slide up and down in the vertical chute is fixedly arranged. A side of the vertical chute away from the stool storage cavity is provided with a pulling cavity. The pulling cavity can slide up and down. A pulling block is provided, and one end of the vertical sliding column away from the sealing plate is rotatably connected to the pulling block, and a spring is fixedly connected between the upper end of the pulling block and the upper wall of the pulling cavity.

9. The vertical self-service blood pressure detector according to claim 8, characterized in that: a reel cavity is provided on the upper side of the pulling cavity, and a reel is rotatably provided between the front and rear walls of the reel cavity, A pulling rope is wound around the reel, the lower end of the pulling rope is fixedly connected to the upper end surface of the pulling block, a driving shaft is fixed on the reel's axis, and an inner wall between the reel chambers is provided. In a synchronization cavity, the opposite end of the transmission shaft extends into the synchronization cavity to be fixed with a transmission bevel gear, and the left end of the telescopic screw extends into the synchronization cavity to be fixed with a power bevel gear that engages the transmission bevel gear.

10. The vertical self-service blood pressure detector according to claim 1, wherein a voice player is provided in the left end surface of the control board and on the upper side of the touch screen.
